(19) United States
(12) Patent Application Publication
(10) Pub. No.: US 2022/0072225 A1
(43) Pub. Date: Mar. 10, 2022

(54) BRANCH ADAPTER FOR INFUSION SET

(71) Applicant: Seong Ki KIM, Busan (KR)

(72) Inventor: Seong Ki KIM, Busan (KR)

(21) Appl. No.: 17/419,795

(22) PCT Filed: Dec. 31, 2019
(86) PCT No.: PCT/KR2019/018763

§ 371 Date: Jun. 30, 2021

(30) Foreign Application Priority Data
Jan. 7, 2019 (KR) 10-2019-0001989

(51) Int. Cl. A61M 5/162; A61M 5/14; A61M 5/168; A61M 39/04

(52) U.S. Cl. CPC A61M 5/162; A61M 5/1418; A61M 2039/1077; A61M 39/045; A61M 5/168

(57) ABSTRACT

The present disclosure provides a branch adapter for an infusion set having a structure in which a valve is not used. More particularly, unlike a conventional technology in which a valve is separately provided such that a branch tube is opened/closed in response to an opening/closing operation of the valve, the present disclosure relates to a branch adapter for an infusion set which provides a structure in which a block-shaped backflow prevention branch tube opening/closing, tool, which is made of an elastic/flexible material such as silicon, is disposed inside a branch tube so as to close the branch tube, while the backflow prevention branch tube opening/closing, tool has a cutting line such that the branch tube is opened when a needle or a connection tube of an infusion tube/a human body-side syringe-connecting tube is inserted through the cutting line, whereby there is no need to provide and use a valve, easiness of manufacture and assembly is improved, manufacturing costs are reduced, and sealing efficiency of the branch tube is improved.

### BACKGROUND OF THE INVENTION

### (a) Field of the Invention

The present disclosure provides a branch adapter for an infusion set having a structure in which a valve is not used. More particularly, unlike a conventional technology in which a valve is separately provided such that a branch tube is opened/closed in response to an opening/closing operation of the valve, the present disclosure relates to a branch adapter for an infusion set which provides a structure in which a block-shaped backflow prevention branch tube opening/closing, tool, which is made of an elastic/flexible material such as silicon, is disposed inside a branch tube so as to close the branch tube, while the backflow prevention branch tube opening/closing, tool has a cutting line such that the branch tube is opened when a needle or a connection tube of an infusion tube/a human body-side syringe-connecting tube is inserted through the cutting line, whereby there is no need to provide and use a valve, easiness of manufacture and assembly is improved, manufacturing costs are reduced, and sealing efficiency of the branch tube is improved.

### (b) Description of the Related Art

Infusion refers to a treatment method or an artificial solution thereof, in which an artificial solution such as a mixture of water and electrolyte, carbohydrate, amino acid solution, glucose solution, plasmanate, or dextran erythrocyte suspension is supplied intravenously or injected into a specific tissue parenterally. In order to perform injection using such an infusion, an infusion set is used. As shown in FIG. 1, an infusion set 10 includes an infusion solution withdrawing chamber 13, which is put in a withdrawal part 12 of an infusion bag 11 where an infusion solution to be administered to a patient is stored and which withdraws the infusion solution; a flow regulator 14 which regulates the injection flow rate of the infusion solution; an infusion tube 15; a connection tube 17 connected to a needle hub 16 to which the needle is connected, and a Y-branch tube 18 for injecting other drugs. Therefore, a needle hub 16 is fixed to a connection tube 17 in a state where the needle inserted into the patient's vein, and then the infusion solution or external drug in the infusion bag 11 is injected into the patient along with the infusion solution.

On the other hand, the infusion set is configured with a three-way valve which regulate so as to administrate the second drug and the like through the injection inflow tube. The three-way valve has a switching handle 110, an infusion inflow tube 102, an injection inflow tube 104, and a discharge tube 106 as shown in FIG. 2. The second drug is flown into the three-way valve through the injection inflow tube 104, is mixed with the infusion solution which is flowed-in through the infusion inflow tube 102, and is discharged through the discharge tube 106.

However, in the case of the conventional technology as described above, there was limitation in that a separate switching valve 20 is provided, and thus, as the branch tube is opened and closed according to the opening/closing operation of the switching valve 20, manufacturing and assembly are cumbersome, and manufacturing costs are increased, and the sealing efficiency of the branch tube is reduced.

### SUMMARY OF THE INVENTION

### Technical Problem

Therefore, the present disclosure has been designed to solve the above-mentioned problems, and it is an object of the present disclosure to provide a new branch adapter for an infusion set having a structure in which a block-shaped backflow prevention branch tube opening/closing tool, which is made of an elastic/flexible material such as silicon, is disposed inside a branch tube so as to close the branch tube, while the backflow prevention branch tube opening closing tool has a cutting line such that the branch tube is opened when a needle or a connection tube of an infusion tube/a human body-side syringe-connecting tube is inserted through the cutting line, whereby there is no need to provide and use a valve, easiness of manufacture and assembly is improved, manufacturing costs are reduced, and sealing efficiency of the branch tube is improved.

### Technical Solution

In order to achieve the above object, the present disclosure provides a branch adapter for an infusion set comprising: an adapter body 100 having a plurality of branch tubes 110; and a backflow prevention branch tube opening/closing tool 200 which is made of an elastic/flexible material, and which is composed of a block body having a shape and size capable of closing the internal passages 140 of the branch tubes 110, and is disposed in the internal passages 140 of the branch tubes 110, and in which a cutting line 210 is penetrated and formed in the direction in which the internal passages 140 of the branch tubes 110 are formed, wherein infusion tubes 200a and 300b inserted into the branch tubes 110 of the adapter body 100, and needles 310 and 410 or connection tubes 320 and 420 of the human body-side syringe-connecting tube 400 are disposed through the cutting line 210 of the backflow prevention branch tube opening/closing tool 200.

In the branch adapter for an infusion set according to the present disclosure as described above, the adapter body 100 may include: a first branch tube 110a to which a first infusion tube 300a is connected; a second branch tube 110b to which the second infusion tube 300b is connected; a third branch tube 110c to which the human body-side syringe-connecting tube 400 is connected; and a central connector 120 to which the first branch tube 110a, the second branch tube 110b, and the third branch tube 110c are connected.

In the branch adapter for an infusion set according to the present disclosure as described above, the backflow prevention branch tube opening/closing tool 200 may be composed of a silicone material.

In the branch adapter for an infusion set according to the present disclosure as described above, the backflow prevention branch tube opening/closing tool 200 comprises: an inlet end 220 which is formed to have the same shape and volume as an inner inlet part of the branch tubes 110 of the adapter body 100, which is closely fixed to the inner inlet part of the branch tubes 110 by force fitting, and through which the cutting line 210 is formed; and an outlet end 230 which is formed to extend from the rear edge part of the inlet end 220 to the rear side and is in close contact with the inner peripheral surface of the branch tubes 110.

### Advantageous Effects

In the branch adapter for an infusion set according to the present disclosure, a block-shaped backflow prevention branch tube opening/closing tool, which is made of an elastic/flexible material such as silicon, is disposed inside a branch tube so as to close the branch tube, while the backflow prevention branch tube opening/closing tool has a cutting line such that the branch tube is opened when a needle or a connection tube of an infusion tube/a human body-side syringe-connecting tube is inserted through the cutting line, whereby there is no need to provide and use a valve, easiness of manufacture and assembly is improved, manufacturing costs are reduced, and sealing efficiency of the branch tube is improved.

### BRIEF DESCRIPTION OF THE DRAWINGS

FIGS. 1 and 2 are views for showing the configuration of a conventional branch adapter for an infusion set;

FIG. 3 is a view for schematically showing the basic configuration of the branch adapter for an infusion set according to the present disclosure;

FIG. 4 is a view for showing that the needle of the infusion tube/body-side syringe connecting tube in the branch adapter for infusion set according to an embodiment of the present disclosure is inserted through the backflow prevention branch tube opening/closing tool;

FIGS. 5 and 6 show examples of using a branch adapter for infusion set according to an embodiment of the present disclosure;

FIG. 7 shows a configuration and operation example of the backflow prevention branch tube opening/closing tool according to an embodiment of the present disclosure;

FIG. 8 is a view for showing that the needle of the infusion tube/body-side syringe connecting tube in the branch adapter for infusion set according to another embodiment of the present disclosure is inserted through the backflow prevention branch tube opening/closing tool;

FIGS. 9 and 10 show examples of using a branch adapter for infusion set according to another embodiment of the present disclosure; and

FIG. 11 shows a configuration and operation example of the backflow prevention branch tube opening/closing tool according to another embodiment of the present disclosure.

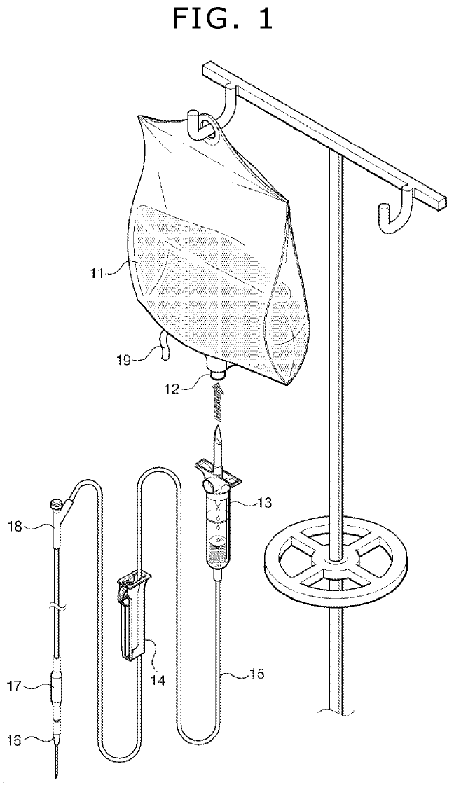

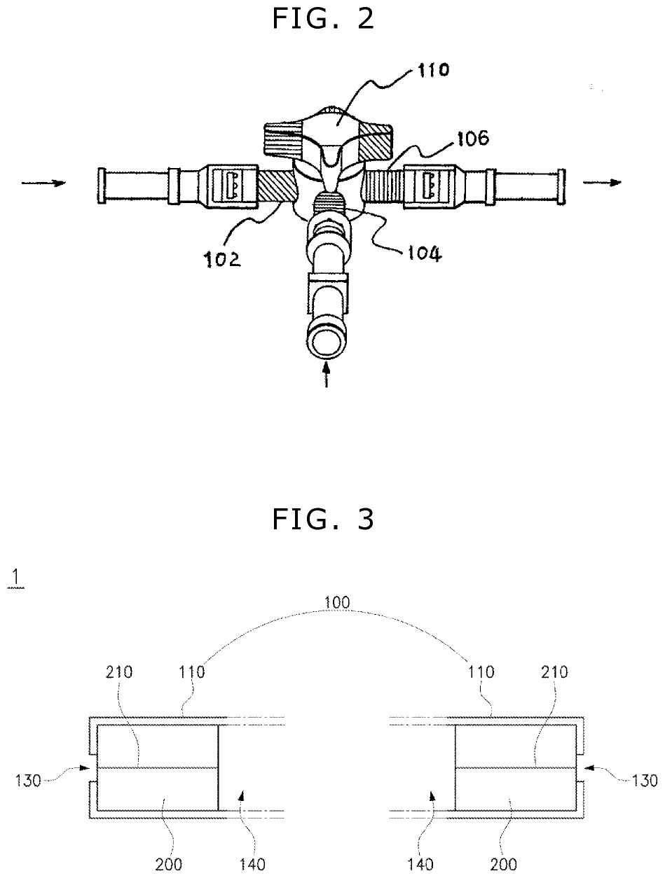

### DETAILED DESCRIPTION OF THE EMBODIMENTS

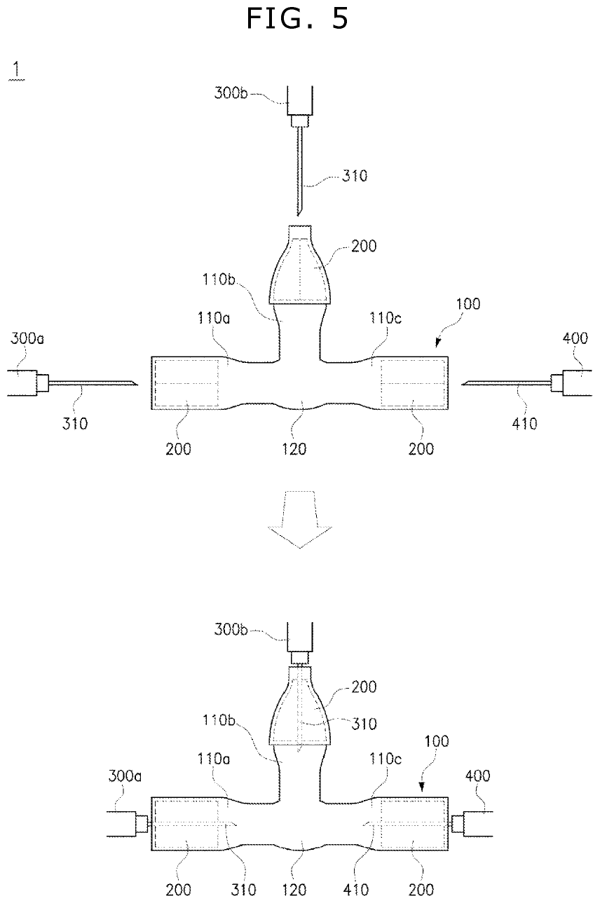

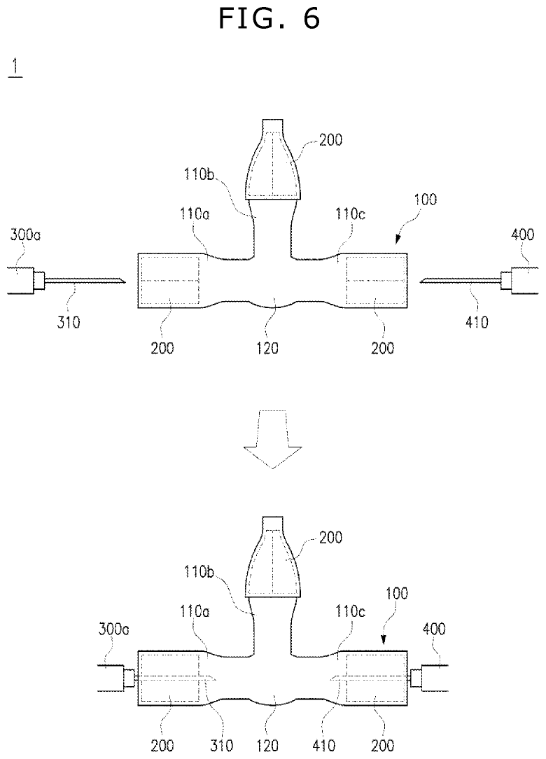

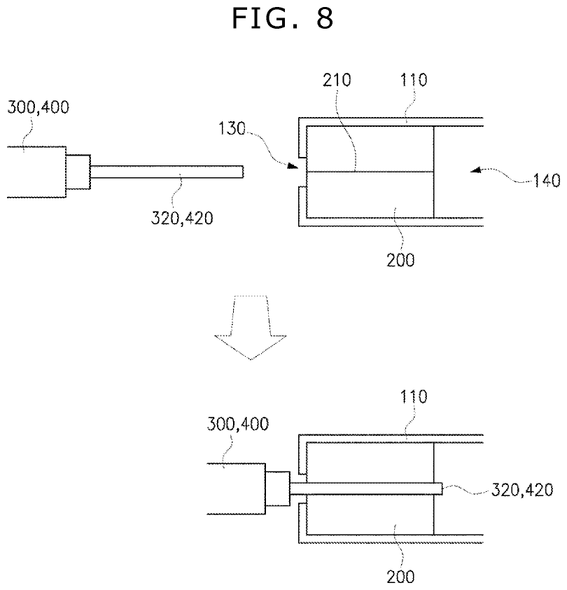

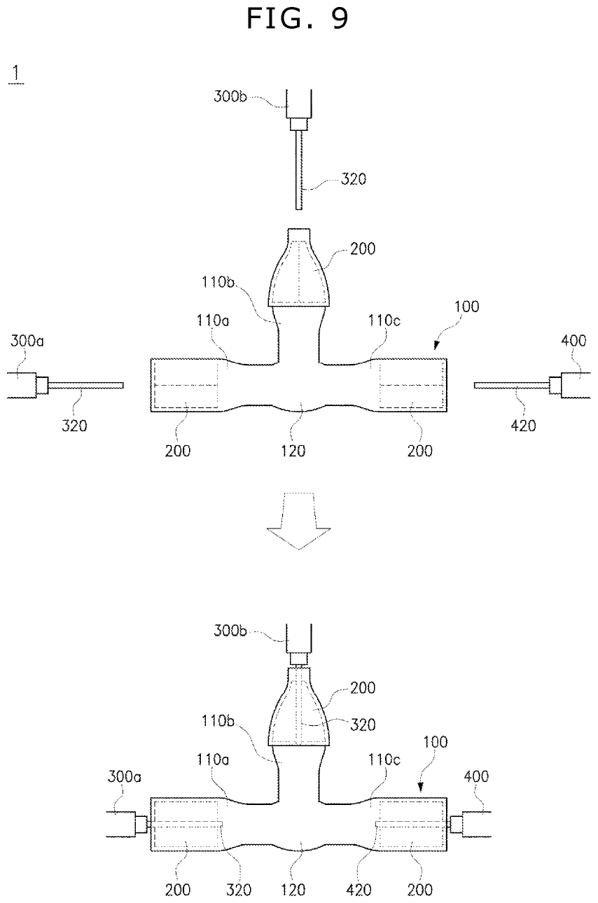

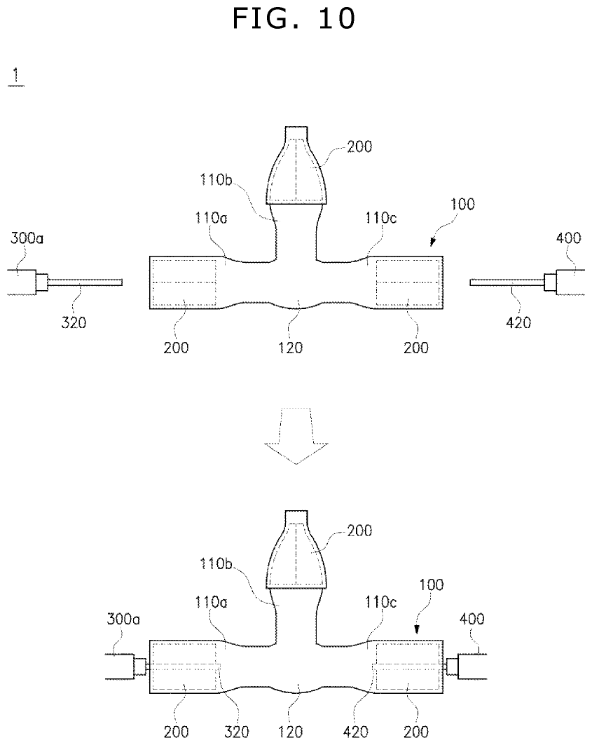

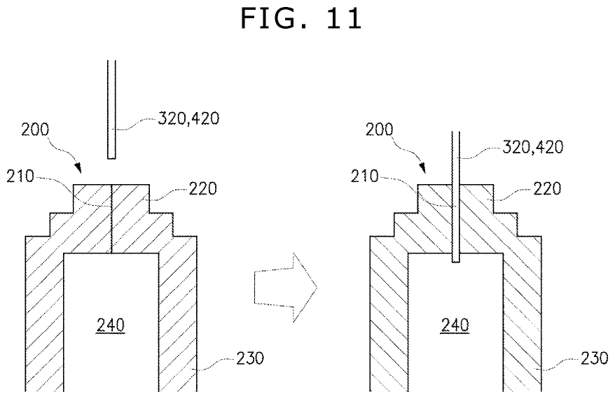

Hereinafter, an embodiment of the present disclosure will be described in detail with reference to FIGS. 3 to 11 of the accompanying drawing. On the other hand, in the drawings and detailed description, the illustration and mention of the construction and operation that can be easily known by those skilled in the art from a general infusion set, a branch adapter for an infusion set, an infusion tube, a human-side syringe connecting tube, a needle, a connection tube, a silicone, etc., the illustration and mention of the configuration and action, which can be easily understood by those in this field, are simplified or omitted. In particular, in the illustration of the drawings and in the detailed description, the description and illustration of specific technical configurations and actions of elements not directly related to the technical features of the present disclosure are omitted, and only the technical configuration related to the present disclosure been briefly illustrated or described.

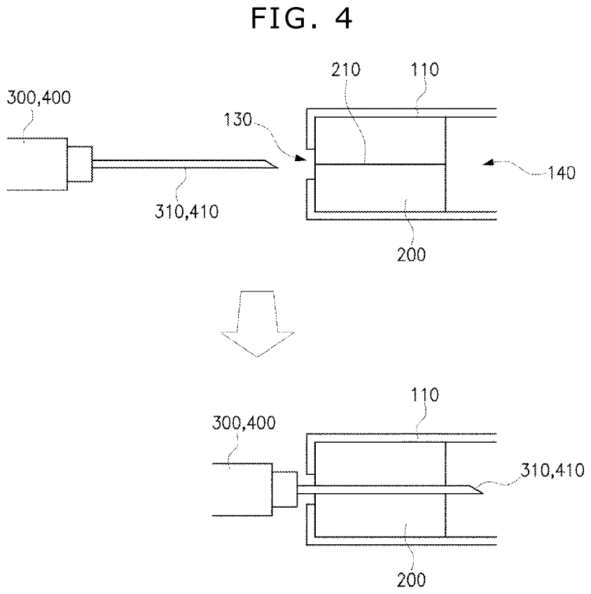

The branch adapter 1 for an infusion set according to an embodiment of the present disclosure includes an adapter body 100 and a backflow prevention branch tube opening/closing tool 200 as shown in FIGS. 3 and 4.

The adapter body 100 has a plurality of branch tubes 110, and the number of branch tubes 110 may be variously implemented.

Here, the adapter body 100 according to an embodiment of the present disclosure includes a first branch tube 110a to which a first infusion tube 300a is connected, a second branch tube 110b to which a second infusion tube 300b is connected, and a third branch tube 110c to which the human body-side syringe-connecting tube 400 is connected, wherein each of the first branch tube 110a, the second branch tube 110b, and the third branch tube 110c consists of a structure in which two or more divided bodies are assembled and combined so that the internal passages 140 are opened.

The backflow prevention branch tube opening/closing tool 200 is made of a material having elasticity and flexibility. In particular, the backflow prevention branch tube opening/closing tool 200 according to the embodiment of the present disclosure is made of a silicone material. Of course, the material of the backflow prevention branch tube opening/closing tool 200 is not limited thereto.

The backflow prevention branch tube opening/closing tool 200 is made of a block body having a shape and size capable of closing the internal passages 140 formed in each branch tube 110 of the adapter body 100, and is disposed in the internal passage 140 of each branch tube 110. To achieve this, the branch tubes 110 having a structure in which two or more divided bodies are assembled and combined is separated, and then the backflow prevention branch tube opening/closing tool 200 is placed in internal passages 140, and then two or more divided bodies are combined and branch tubes 110 are formed again.

The internal passage 140 of each branch tube 110 is closed by such a backflow prevention branch tube opening/closing tool 200. And the backflow prevention branch tube opening/closing tool 200 is formed through the cutting line 210 in the direction in which the internal passage 140 of each branch tube 110 is formed. The cutting line 210 is formed without a gap so that the backflow prevention branch tube opening/closing tool 200 can close the internal passage 140 of each branch tube 110.

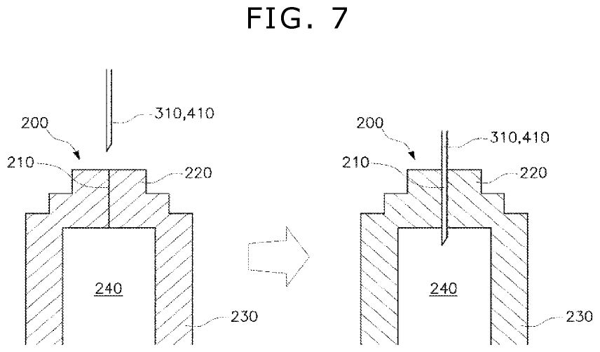

Here, the backflow prevention branch tube opening/closing tool 200 according to the embodiment of the present disclosure is configured so as to include an inlet end 220 and an outlet end 230 as shown in FIG. 7.

The inlet end 220 is formed to have the same shape and volume as the inner inlet part of the branch tubes 110 of the adapter body 100, and is tightly fixed to the inner inlet part of the branch tubes 110 by force fitting. The cutting line 210 is formed through the inlet end 220 as described above.

The outlet end 230 is formed so as to extend from the rear edge part of the inlet end 220 to the rear side and is in close contact with the inner peripheral surface of the branch tubes 110. In the inside of the outlet end 230, an inner space 240 is formed in which the infusion tubes 200a and 300b and the tips of the needles 310 and 410 or connectors 320 and 420 of the human-body side syringe connecting tube 400 are located.

In the branch adapter 1 for an infusion set according to an embodiment of the present disclosure configured in this manner, an infusion tubes 200a and 300b inserted into the inlet 130 of the branch tubes 110 of the adapter body 100 and the needles 310 and 410 or the connection tube 320 of the syringe connecting tube 400 on the human body side 420 is arranged to penetrate through the cutting line 210 of the backflow prevention branch tube opening/closing tool 200.

In the branch adapter 1 for an infusion set according to an embodiment of the present disclosure configured in this manner, a block-shaped backflow prevention branch tube opening/closing tool 200, which is made of an elastic/flexible material such as silicon, is disposed inside a branch tube 110 so as to close the branch tube, while the backflow prevention branch tube opening/closing tool 200 has a cutting line such that the branch tubes 110 are opened when a needle or a connection tube of an infusion tube/a human body-side syringe-connecting tube is inserted through the cutting line, whereby there is no need to provide and use a valve, easiness of manufacture and assembly is improved, manufacturing costs are reduced, and sealing efficiency of the branch tube is improved.

As described above, although the branch adapter for the infusion set according to the embodiment of the present disclosure is shown according to the above description and drawings, it will be understood by those skilled in the art that various changes and modifications can be made without departing from the spirit and scope of the present disclosure.

What is claimed is:

1. A branch adapter for an infusion set comprising:
an adapter body 100 having a plurality of branch tubes 110; and
a backflow prevention branch tube opening/closing tool 200 which is made of an elastic/flexible material, and which is composed of a block body having a shape and size capable of closing the internal passages 140 of the branch tubes 110, and is disposed in the internal passages 140 of the branch tubes 110, and in which a cutting line 210 is penetrated and formed in the direction in which the internal passages 140 of the branch tubes 110 are formed,
wherein infusion tubes 200a and 300b inserted into the branch tubes 110 of the adapter body 100, and needles 310 and 410 or connection tubes 320 and 420 of the human body-side syringe-connecting tube 400 are disposed through the cutting line 210 of the backflow prevention branch tube opening/closing tool 200.

2. The branch adapter for an infusion set as claimed in claim 1, wherein:
the adapter body 100 comprises:
a first branch tube 110a to which a first infusion tube 300a is connected;
a second branch tube 110b to which a second infusion tube 300b is connected;
a third branch tube 110c to which the human body-side syringe-connecting tube 400 is connected; and
a central connector 120 to which the first branch tube 110a, the second branch tube 110b, and the third branch tube 110c are connected.

3. The branch adapter for an infusion set as claimed in claim 1, wherein:
the backflow prevention branch tube opening/closing tool 200 is made of a silicone material.

4. The branch adapter for an infusion set as claimed in claim 1, wherein:
the backflow prevention branch tube opening/closing tool 200 comprises:
an inlet end 220 which is formed to have the same shape and volume as an inner inlet part of the branch tubes 110 of the adapter body 100, which is closely fixed to the inner inlet part of the branch tubes 110 by force fitting, and through which the cutting line 210 is formed; and
an outlet end 230 which is formed to extend from the rear edge part of the inlet end 220 to the rear side and is in close contact with the inner peripheral surface of the branch tubes 110.
